(19) United States
(12) Patent Application Publication
(10) Pub. No.: US 2026/0274770 A1
(43) Pub. Date: Sep. 17, 2026

(54) DEPOLYMERIZATION OF CROSS-LINKED POLYETHYLENE

(71) Applicant: Basell Poliolefine Italia S.r.l., Milano (IT)

(72) Inventors: Shahram Mihan, Frankfurt/M (DE); Volker Fraaije, Frankfurt/M (DE); Gerd Mannebach, Frankfurt/M (DE); Holger Bruening, Frankfurt/M (DE); Christof Justus, Frankfurt/M (DE); Hans Leibold, Karlsruhe (DE); Frank Richter, St. Ingbert (DE)

(73) Assignee: Basell Poliolefine Italia S.r.l., Milano (IT)

(21) Appl. No.: 19/168,689

(22) PCT Filed: Mar. 27, 2024
(86) PCT No.: PCT/EP2024/058263

§ 371 Date: Sep. 24, 2025

(30) Foreign Application Priority Data
Mar. 30, 2023 (EP) 23165471.6

(51) Int. Cl. C07C 4/22

(52) U.S. Cl. CPC C07C 4/22

(57) ABSTRACT

A method for producing a cracker feedstock by depolymerizing plastic waste material made from or containing cross-linked polyethylene. A method for producing a cracker feedstock by depolymerizing a polymeric waste material comprising crosslinked polyethylene (PEX), comprising the steps of: (i) introducing the waste material into a pyrolysis reactor; (ii) mixing the waste material with a depolymerization catalyst comprising an acidic compound; (iii) depolymerizing the waste material in an oxygen-free atmosphere at a temperature ranging from 400 to 590° C., thereby obtaining a gaseous depolymerization product; and (iv) collecting and condensing at least part of the gaseous depolymerization product, thereby obtaining the cracker feedstock.

### FIELD OF THE DISCLOSURE

In general, the present disclosure relates to the field of chemistry. More specifically, the present disclosure relates to polymer chemistry. In particular, the present disclosure relates to a method for producing a cracker feedstock by depolymerizing cross-linked polyethylene, a multicomponent material made from or containing polyolefins, or both.

### BACKGROUND OF THE DISCLOSURE

In some instances, crosslinked polyethylene is used in multicomponent materials. In some instances, crosslinked polyethylene is abbreviated as PEX, XPE or XLPE. In some instances, the applications include building service pipework systems, hydronic radiant heating or floor heating and cooling systems, domestic water piping, and insulation for high tension electrical cables. In some instances, crosslinked polyethylene is used in natural gas and offshore oil applications, district heating, chemical transportation, transportation of sewage and slurries, and mining.

In some instances, cross-linked polyethylene provides flexibility and low costs as well as easier installation than other materials. In some instances, crosslinked polyethylene is easier to install than copper for piping applications. In some instances, crosslinked polyethylene's longevity is due to characteristics of non-corrosion and stress crack and abrasion resistance. In some instances, pipes made from or containing crosslinked polyethylene are candidates for replacing metal and thermoplastic pipes.

In some instances, recycling of cross-linked polyethylene is challenging because the material is shredded and compounded together with non-crosslinked material, thereby preventing use of the resulting material in standard applications. It is believed that the ultra-high molecular weight of the crosslinked polymer chains prevents homogenous mixing and yields large gels. As such and in some instances, mechanical recycling is not an option.

### SUMMARY OF THE DISCLOSURE

In a general embodiment, the present disclosure provides a method for producing a cracker feedstock by depolymerizing polymeric waste material made from or containing crosslinked polyethylene, including the steps of:
i) introducing the waste material into a pyrolysis reactor;
ii) mixing the waste material with a depolymerization catalyst;
iii) depolymerizing the waste material in an oxygen-free atmosphere, thereby obtaining a gaseous depolymerization product; and
iv) collecting and condensing at least part of the gaseous depolymerization product, thereby obtaining the cracker feedstock.
   In some embodiments, the pyrolysis reactor is a reactor with a screw conveyer.

In some embodiments, the catalyst is a particulate catalyst made from or containing an acidic compound as the active component. In some embodiments, the acidic compound is deposited on a particulate non-porous support. In some embodiments, the acidic compound is deposited with the aid of a coating agent.

In some embodiments, the acidic compound is selected from the group consisting of Al/Si mixed oxides, Al₂O₃, aluminosilicates, silica and zeolites.

In some embodiments, the particulate non-porous support is selected from the group consisting of sand, glass beads and metal particles.

In some embodiments, the coating agent is selected from the group consisting of oil, inorganic hydrogel and combinations thereof.

In some embodiments, the waste material is further made from or containing aluminum. In some embodiments, the method further includes a step of: collecting the aluminum from the waste material.

In some embodiments, the crosslinked polyethylene is selected from the group consisting of peroxide-crosslinked polyethylene, silane-crosslinked polyethylene, irradiation-crosslinked polyethylene, radically crosslinked polyethylene, azo-crosslinked polyethylene, UV initiated radical crosslinked polyethylene and mixtures thereof.

In some embodiments, the waste material is obtained from PEX pipes, PEX/Al/PEX pipes, Al/PEX pipes, PEX/Al/PEX pipes, or combinations thereof.

In some embodiments, the polymeric waste material is made from or containing, alternatively consisting of, shredded PEX pipe waste and plastic waste stream with a melt index (MFI, 21.6) of higher than 10 g/10 min. In some embodiments, the polymeric waste material is made from or containing at least 40 wt.-%, alternatively 40 to 80 wt.-%, of the plastic waste stream, based on the total weight of the waste material.

In some embodiments, the polymeric waste material is made from or containing a polymeric fraction made from or containing a low amount, alternatively an amount lower than 12 wt. %, alternatively an amount less than 10 wt. %, of non-polyolefin components, based upon the total weight of the dry weight polymeric fraction.

In some embodiments, the gaseous depolymerization product is released from the reactor by gas release units placed along a screw conveyer.

In some embodiments, the collection of the gaseous depolymerization product includes a step of hot gas filtration.

In some embodiments, condensing of the gaseous depolymerization product is achieved by sequentially passing the gaseous depolymerization product through a plurality of condensing units.

In some embodiments, the residence time of the waste material in the reactor is no more than 60 minutes, alternatively no more than 45 minutes.

In some embodiments, the reactor is operated at a temperature of 350 to 650° C., alternatively 400 to 590° C., alternatively from 430 to 500° C.

In some embodiments, the reactor is operated at a pressure of 0.7 to 10 bar_{g}.

In some embodiments, the reactor is heated electrically.

In some embodiments, at least part of the non-liquefiable depolymerization product (if present), at least part of the catalyst, or both are re-introduced into the reactor.

In some embodiments, the method further includes the step of monitoring the content of methane in the collected gaseous depolymerization product.

In some embodiments, the collected depolymerization product is made from or containing more than 60 wt.-% of gaseous components, based on the total weight of the depolymerization product. In some embodiments, the gaseous components are made from or containing more than 50 wt.-% of olefins, based on the total weight of the gaseous components.

In some embodiments, the liquid depolymerization product is made from or containing from 35 to 45 wt.-% of a high boiling fraction, from 40 to 50 wt.-% of a medium boiling fraction, and from 15 to 25 wt.-% of a low boiling fraction.

### DETAILED DESCRIPTION OF THE DISCLOSURE

In some embodiments, the present disclosure provides a method for producing a cracker feedstock by depolymerizing polymeric waste material made from or containing crosslinked polyethylene, using an acidic depolymerization catalyst.

In a first step of the method, the polymeric waste material is introduced into a pyrolysis reactor. In some embodiments, the pyrolysis reactor is a reactor with a screw conveyer. In some embodiments, the screw conveyer is used in combination with the particulate catalyst. In some embodiments, a homogenous mixture of waste material and catalyst was obtained, thereby achieving a homogeneous heating of the waste material. It is believed that the catalyst acts as heat carrier and prevents the molten waste material from sticking to the screw.

In some embodiments and to assist in pyrolyzing and depolymerizing the polymeric waste material, the catalyst is in particulate form. In some embodiments, the particulate non-porous support is selected from the group consisting of sand, glass beads and metal particles. In some embodiments, the particulate non-porous support has a variety of shapes. In some embodiments, the shape is selected from the group consisting spherical, cylindrical, and non-homogenous shapes. In some embodiments, the support is also non-porous. As used herein, the term “non-porous” refers to the state of being not permeable to air, water or other liquids.

In some embodiments, the catalyst is designed for mixing with the polymeric waste material undergoing depolymerization. In some embodiments and to ensure mixing, the catalyst has an average particle size of 0.2 to 20 mm, alternatively 0.5 to 10 mm, determined according to Coulter counter analysis in accordance with ASTM D4438. In some embodiments, the non-porous particulate support has a particle size distribution of

| | Min | Max |
|---|---|---|
| <3000 μm | 70% | 90% |
| >1400 μm | 40% | 80% |
| >1000 μm | 50% | 80% |
| <1000 μm | 0% | 20% |

In some embodiments, the non-porous particulate support is sand.

In some embodiments, the acidic compound of the catalyst is selected from the group consisting of Al/Si mixed oxides, Al₂O₃, aluminosilicates, silica and zeolites. As used herein, the term “Al/Si mixed oxides” refers to a material made from or containing a mixture of Al₂O₃ and SiO₂, having a neutral structure.

As used herein, the term “zeolites” refers to crystalline microporous aluminosilicates, which are built up from corner-sharing SiO₄⁻ and AlO₄⁻ tetrahedrons, having the structure Mⁿ⁺_{x/n} [AlO₂]⁻ₓ(SiO₂)_{y}]+zH₂O with n being the charge of the cation M and z defining the number of water molecules incorporated into the crystal structure. In some embodiments, M is an alkaline, alkaline earth metal, or hydrogen ion. In some embodiments, the ion is selected from the group consisting of H⁺, Na⁺, Ca²⁺, K⁺, and Mg²⁺. As described herein, zeolites differ from mixed Al/Si oxides by the zeolites' defined pore structure and ionic character. In some embodiments, the zeolite employed as the acidic compound is selected from the group consisting of Zeolite Y, Zeolite Beta, Zeolite A, Zeolite X, Zeolite L and mixtures thereof, alternatively Zeolite Y and Zeolite Beta. In some embodiments, zeolites have the metal ion M substituted by a hydrogen. In some embodiments, the zeolite-type components are selected from the group consisting of ZSM-5, ZSM-11, ZSM-22, ZSM-23, ZSM-35, ZSM-48, ZSM-50, TS-1, TS-2, SSZ-46, MCM-22, MCM-49, FU-9, PSH-3, ITQ-1, EU-1, NU-10, silicalite-1, silicalite-2, boralite-C, boralite-D, BCA, and mixtures thereof.

In some embodiments, the acidic compound is an Al/Si mixed oxide. In some embodiments, the composition of the Al/Si mixed oxide is adjusted. In some embodiments, the acidic compound is made from or containing Al₂O₃ in an amount from 20 to 99 wt. %, alternatively from 30 to 80 wt. %, alternatively from 40 to 70 wt. %, based on the total weight of the acidic compound. In some embodiments, the acidic compound is made from or containing SiO₂ in an amount from 1 to 80 wt. %, alternatively from 20 to 70 wt. %, alternatively from 30 to 60 wt. %, based on the total weight of the acidic compound.

In some embodiments, the acidic compound contains a slight excess of Al₂O₃, thereby improving the depolymerization. In some embodiments, the acidic compound is made from or containing an excess of Al₂O₃. In some embodiments, the weight ratio of Al₂O₃ to SiO₂ in the acidic compound is from 99:1 to 30:70, alternatively from 9:1 to 3:2, alternatively from 4:1 to 3:2.

In some embodiments, the determination of the SiO₂ and Al₂O₃ content of the acidic compound is carried out by atomic emission spectroscopy, using an inductively coupled plasma (ICP-AES).

In some embodiments, the coating agent is selected from the group consisting of oil, inorganic hydrogel or combinations thereof. In some embodiments, the inorganic hydrogel is a silica hydrogel. In some embodiments, the oil is aromatic-free white mineral oil. In some embodiments, the aromatic-free white mineral oil is based on iso-paraffins. In some embodiments, the oil has a kinematic viscosity at 20° C. of 140 to 180 mm²/s, alternatively 150 to 170 mm²/s. In some embodiments, the oil has a kinematic viscosity at 40° C. of 40 to 80 mm²/s, alternatively 50 to 70 mm²/s. In some embodiments, the oil has a kinematic viscosity at 100° C. of 5 to 15 mm²/s, alternatively 7 to 10 mm²/s. In some embodiments, the kinematic viscosity is determined according to ISO 3104.

In some embodiments, the catalyst is made from or containing 0.5 to 6 wt.-%, alternatively 2 to 4 wt.-%, of the active compound, based on the total weight of the catalyst.

In some embodiments, the catalyst is obtained by mixing the particulate non-porous support and the coating agent, thereby obtaining a mixture, and then adding the acidic compound in the form of a powder to the resulting mixture. In some embodiments, the mixture is heat treated, thereby obtaining the catalyst. In some embodiments, the heat treatment is carried out at a temperature of 100 to 600° C. In some embodiments, the particulate non-porous support is subjected to a drying step before being mixed with the coating agent.

In some embodiments, the catalyst is obtained by a process including the steps of:
a) mixing the particulate non-porous support and the coating agent, thereby yielding a mixture; and
b) adding the acidic compound in powder form to the mixture of step a).
   In some embodiments, the catalyst is made from or containing:
sand as a particulate non-porous support;
an Al/Si mixed oxide or a zeolite as an acidic compound; and
mineral oil or silica hydrogel as a coating agent.

In some embodiments, the catalyst is reactivated by heating, thereby allowing multiple uses and conserving resources. In some embodiments, the heat treatment is carried out at a temperature of 500 to 900° C., alternatively 600 to 900° C., alternatively 550-850° C. In some embodiments, the heat treatment is carried out in an oxidative atmosphere such as air or oxygen. In some embodiments, treatment conditions are selected based upon catalyst activity and energy consumption, using carbon residue concentration on the catalyst as an indicator. In some embodiments, carbon residue of regenerated catalyst is less than 20 weight % of the catalyst, alternatively less than 15%, alternatively less than 10%, alternatively less than 5%. In some embodiments, treatment times vary. In some embodiments, the treatment times are between 0.5 to 100 hours, alternatively 1 to 20 hours, alternatively 2 to 10 hours.

In some embodiments, the waste material is made from or containing waste crosslinked polyethylene. In some embodiments, the waste material is selected from different kinds of crosslinked polyethylene.

In some embodiments, the crosslinked polyethylene in the polymeric waste material is selected from the group consisting of peroxide-crosslinked polyethylene, silane-crosslinked polyethylene, irradiation-crosslinked polyethylene, radically crosslinked polyethylene, azo-crosslinked polyethylene, UV initiated radical crosslinked polyethylene and mixtures thereof.

In some embodiments, the polymeric waste material is derived from various applications. In some embodiments, those applications include pipe and cable applications, alternatively high-voltage cables. Ins some embodiments, the polymeric waste material is made from or containing crosslinked polyethylene and multicomponent materials. In some embodiments, the multicomponent materials are made from or containing polyolefins. In some embodiments, pipes and cables are made from or containing multiple layers, having a layer made from or containing crosslinked polyethylene. In some embodiments, the multicomponent materials are further made from or containing materials selected from the group consisting of metals, adhesives and other polymers. In some embodiments, the metal is aluminum. In some embodiments, the other polymer is EVOH. In some embodiments, the polymeric waste material is obtained from multicomponent material made from or containing crosslinked PE. In some embodiments, the polymeric waste material is obtained from multicomponent material made from or containing high voltage cables made from or containing PEX, PEX pipes, PEX/Al/PEX pipes, PEX/Al/PE-RT pipes, PEX/Al/PE pipes, or combinations thereof.

In some embodiments, the waste material is made from or containing, alternatively consisting of, at least one of the following:
i) 5-layer Pipe: PEX/Ad/Alu/Ad/PEX′
ii) 5-layer Pipe: PEX/Ad/Alu/Ad/PE;
ii) 5-layer Pipe: PEX/Ad/Alu/Ad/PERT;
iv) 5-layer Pipe: PEX/Ad/EVOH/Ad/PEX;
v) 5-layer Pipe: PEX/Ad/EVOH/Ad/PE;
vi) 5-layer Pipe: PEX/Ad/EVOH/Ad/PERT;
vii) 3-layer Pipe: PEX/Ad/EVOH;
viii) 3-layer Pipe: stainless steel/Ad/PEX; and
ix) 3-layer Pipe: copper/Ad/PEX, wherein
   PEX refers to a layer made from or containing crosslinked polyethylene, Ad refers to a layer made from or containing an adhesive, Alu refers to a layer made from or containing aluminum, PE-RT refers to a layer made from or containing polyethylene for raised temperature applications, PE refers to a layer made from or a containing polyethylene and EVOH refers to a layer made from or containing ethylene vinyl alcohol.

In some embodiments, the polymeric waste material is made from or containing, alternatively consisting of, a blend made from or containing shredded PEX pipe waste and plastic waste stream with a melt index (MFI/21.6) of higher than 10 g/10 min. In some embodiments, the polymeric waste material is made from or containing of at least 40 wt.-% of the waste material, alternatively 40 to 80 wt.-%, of the plastic waste stream, based on the total weight of the waste material.

In some embodiments, the polymeric waste material has a bulk density of at least 150 g/cm³. It is believed that the value of bulk density facilitates a continue flowless depolymerization process, prevents blockage of feeding lines, and prevents reactor fouling. In some embodiments, the method provides low amounts of residues, thereby enhancing the depolymerization reaction and increasing the yield of depolymerization products. In some embodiments, the polymeric waste material has a bulk density of 200 to 700 g/cm³, alternatively 200 to 600 g/cm³, alternatively 250 to 550 g/cm³, determined according to DIN 53466.

In some embodiments, the polymeric waste material is a shredded pipe with a particle size of <50 mm, alternatively <30 mm, alternatively <20 mm, alternatively <15 mm.

In some embodiments, the polymeric waste material is a shredded pipe with a bulk density of at least 300 g/cm³, a particle size of <20 mm, and PE+PP content of at least 90% of organic content.

In some embodiments, the waste material is made from or containing crosslinked polyethylene and aluminum. In some embodiments, the method further includes a step of: collecting the aluminum from the waste material feedstock.

In some embodiments, the method yields a liquid depolymerization product. In some embodiments, the liquid depolymerization product is employed as feedstock for crackers in the production of ethylene. In some embodiments, the gaseous depolymerization product is released from the screw reactor by gas release units, which are placed along the screw conveyer. In some embodiments, different vapor fractions are sequentially released along the screw and collected individually. In some embodiments, the gaseous depolymerization product is condensed to a liquid depolymerization product.

In some embodiments, the method produces a highly selective depolymerization product. In some embodiments, the selectivity determined by the combination of screw reactor and particulate catalyst. In some embodiments, selectivity is determined by hot gas filtration. In some embodiments, hot gas filtration is combined with collecting the gaseous depolymerization product. In some embodiments, collection of the gaseous depolymerization product includes a step of hot gas filtration.

In some embodiments, the gaseous depolymerization product is at least partially condensed, thereby yielding a liquid depolymerization product. In some embodiments, the liquid depolymerization product is further processed as cracker feedstock. In some embodiments, condensing of the gaseous depolymerization product is achieved by sequentially passing the gaseous depolymerization product through a plurality of condensing units. In some embodiments, the condensing units are operated at different temperatures, thereby facilitating separation of the high boiling fraction, the medium boiling fraction, and the high boiling fraction of the depolymerization product.

In some embodiments, the method allows for a comparatively short residence time of the waste material in the reactor. In some embodiments, the residence time of the waste material is no more than 60 minutes, alternatively no more than 45 minutes.

In some embodiments, the method is conducted low temperatures. In some embodiments, the combination of low temperatures and short residence time preserves energy and reduces the carbon footprint. In some embodiments, the reactor is operated at a temperature of 350 to 650° C., alternatively 400 to 590° C., alternatively from 430 to 500° C. In some embodiments, the reactor is operated at a pressure of 0.7 to 10 bar_{g}.

In some embodiments, the reactor is heated electrically. In some embodiments, heating supports detailed mass and heat balancing of the depolymerization process, thereby allowing for selectivity. In some embodiments, heating is combined with sequential release of the different vapor fractions along the screw conveyor, thereby supporting detailed mass and heat balancing and allowing for selectivity.

In some embodiments, the depolymerization product is further made from or containing non-liquifiable fractions. In some embodiments, those non-liquifiable fractions are re-introduced into the reactor, thereby minimizing the production of non-usable depolymerization waste. In some embodiments, the catalyst is employed in more than one cycle. In some embodiments, the catalyst is re-activated. In some embodiments, at least part of the catalyst is re-introduced into the reactor.

In some embodiments, it is believed that the content of methane in the gaseous depolymerization indicates the progress of depolymerization. In some embodiments, the content of methane in the collected gaseous depolymerization product is monitored.

In some embodiments, the method yields selectively a depolymerization product. In some embodiments, the liquid depolymerization product is made from or containing from 35 to 45 wt.-% of a high boiling fraction, from 40 to 50 wt.-% of a medium boiling fraction and from 15 to 25 wt.-% of a low boiling fraction.

In some embodiments, the method generates little to no char. In some embodiments, the residue of the depolymerization method has a char content of less than 5 wt. %, alternatively less than 2 wt. %, based on the total weight of the depolymerization product.

In some embodiments, the liquid depolymerization product is further separated. In some embodiments, the method further includes a step of: distilling the liquid depolymerization product.

In some embodiments, the method yields a depolymerization product with a high gaseous content. In some embodiments, the gaseous content in the depolymerization product after step iv) is more than 30 wt.-%, alternatively more than 50 wt.-%, alternatively more than 60 wt.-%, alternatively more than 70 wt.-%, of gaseous components, based on the weight of polymeric fraction of the polymeric waste material. In some embodiments, the gaseous components are made from or containing more than 50 wt.-% of olefins, based on the total weight of the gaseous components.

In some embodiments, the gaseous fraction of the depolymerization product has a high content of monomeric olefinic C₂-C₄-compounds. In some embodiments, the monomeric olefinic C₂-C₄-compounds are used for producing polymers. In some embodiments and in view of the amount of monomeric olefinic C₂-C₄-compounds generated during depolymerization, the gaseous depolymerization product is directly used as feedstock in cracking processes and subsequent polymerization. In some embodiments, the gaseous depolymerization product is made from or containing light olefins and light alkanes. In some embodiments, the gaseous depolymerization product is transferred to a downstream cracker by passing the ovens to produce polymerization grade monomer streams. In some embodiments, embodiments, other side products are cracked in the oven. In some embodiments, the side products are selected from the group consisting of ethane, propane and butanes. In some embodiments, the steps of treating the depolymerization product to obtain monomers is bypassed, thereby saving energy and reducing CO₂ output.

### i) Liquid Depolymerization Product

In some embodiments, the resulting liquid depolymerization product has a low content of aromatic compounds, alternatively a low content of polycyclic aromatic compounds and asphaltanes. In some embodiments, the liquid depolymerization product has a low content of aromatic and olefinic components and a high degree of purity.

In some embodiments, the liquid depolymerization product has less than 10 mol %, alternatively less than 5 mol %, alternatively no more than 3 mol %, of aromatic components, measured as contents of aromatic protons in mol % as determined by ¹H-NMR-spectroscopy.

In some embodiments, the liquid depolymerization product has a low content of olefinic compounds. In some embodiments, the liquid depolymerization product has less than 5 mol %, alternatively less than 3 mol %, alternatively less than 1.5 mol %, alternatively no more than 1 mol %, of olefinic compounds, determined based on the contents of olefinic protons, based on the total number of hydrocarbon protons, as determined by ¹H-NMR-spectroscopy.

In some embodiments, the content of double bonds is measured by the Bromine number (BrNo.), which indicates the degree of unsaturation. In some embodiments, the liquid depolymerization product has a Bromine number, expressed as gram bromine per 100 grams, of less than 25, alternatively from 0.1 to 20, alternatively from 0.2 to 15, alternatively from 0.3 to 10, alternatively from 0.5 to 5, determined according to ASTM D1159-01.

In some embodiments, the liquid depolymerization product has a boiling range from 30 to 650° C., alternatively from 50 to 250° C. In some embodiments, the depolymerization product is separated into hydrocarbon fractionations of different boiling ranges. In some embodiments, the hydrocarbon fractions are selected from the group consisting of a light naphtha fraction made from or containing C₅ and C₆ hydrocarbons having a boiling range from 30° C. and 130° C., a heavy naphtha fraction made from or containing C₆ to C₁₂ hydrocarbons having a boiling range from 130° C. to 220° C., a kerosene fraction made from or containing C₉ to C₁₇ hydrocarbons having a boiling range from 220° C. to 270° C., and other high boiling point fractions. In some embodiments, the other high boiling point fractions are selected from the group consisting of diesel fuel, fuel oil, and hydrowax. In some embodiments, the separation occurs by distillation.

In some embodiments, the liquid depolymerization product has little to no solid residue. In some embodiments and upon evaporation of the liquid depolymerization product, the content of residues is no more than 5 ppm (w), determined according to ASTM D381.

### ii) Gaseous Depolymerization Product

In some embodiments, the gaseous depolymerization product has a low content of low molecular hydrocarbons. In some embodiments, the low molecular hydrocarbons are methane or ethane. In some embodiments, the gaseous depolymerization product is made from or containing high amounts of higher olefins. In some embodiments, the higher olefins are selected from the group consisting of ethylene propylene and butenes. In some embodiments, the gaseous depolymerization product is made from or containing (i) a high content of ethylene, propylene, butenes, or a combination thereof, (ii) a low content of saturated low molecular hydrocarbons, alternatively hydrocarbons of the formula CₙH₂ₙ₊₂ wherein n is a real number ranging from 1 to 4, or (iii) a combination thereof.

In some embodiments, the gaseous depolymerization product has a content of methane of at most 5 wt.-%, alternatively at most 4 wt.-%, alternatively at most 3 wt.-%, alternatively at most 2 wt.-%, alternatively at most 0.5-1.5 wt.-%, based on the total weight of the gaseous depolymerization product.

In some embodiments, the gaseous depolymerization product is made from or containing a high amount of low molecular olefinic compounds. In some embodiments, the low molecular olefinic compounds are the CₙH₂ₙ variety with n=2-4. In some embodiments, the gaseous fraction is directly used as feedstock for further processing in a cracker downstream, alternatively a raw gas compressor, thereby obtaining purified monomer streams, and thereafter for the subsequent production of polymers, thereby allowing for bypassing the stream cracking ovens and reducing the output of CO₂. In some embodiments, the gaseous depolymerization product is made from or containing at least 50 wt.-%, alternatively at least 60 wt.-%, alternatively at least 65 wt.-%, alternatively at least 70 wt.-%, alternatively at least 75 wt.-%, of compounds of the formula CₙH₂ₙ (olefins) with n=2-4, based on the total weight of the gaseous depolymerization product.

In some embodiments, the gaseous depolymerization product has small quantities of HCl, HCN, H₂S, H₂O, NH₃, or COS. In some embodiments, the quantities of HCl, HCN, H₂S, H₂O, NH₃, or COS are separated in a refining step before introduction to the steam cracker downstream segments.

The method of the present disclosure will be explained in more detail with reference to the following examples which are not intended to limit the scope and spirit of the disclosure.

The following analytical methods were employed:
1) GC MS was used for liquid and gas analysis.
2) Char residue was determined according to mass balance after decoking the residues of the reactor at 800° C.
3) Liquid contents were characterized using simulated distillation (SimDist) analysis according to ASTM D 7213:2012.
4) The total content of unsaturated components in the liquid condensates were characterized via Bromine number determination using an 848 Titrino Plus (Metrohm AG, Herisau, Switzerland), equipped with a double PT-wire electrode, which had integrated PT1000 temperature sensor, and a 10 ml burette, in accordance with ASTM D1159-01 as described in Metrohm Application Bulletin 177/5e, December 2018. The Bromine number (BrNo.) represented the amount of bromine in grams absorbed by 100 grams of a sample.
5) ¹H-NMR analysis was conducted by dissolving a sample of the liquid condensate in CDCl₃ and characterizing the sample using proton NMR spectroscopy. Aromatic, olefinic and aliphatic protons were assigned according to the chemical shifts summarized in Table 1:

TABLE 1
Integral Regions in ¹H-NMR spectroscopy

| Peak Assignment | ¹H Chemical Shift (ppm) |
|---|---|
| I₁ (Aromatic Protons) | 8.25-7.27 |
| CDCl₃-Solvent | 7.26 |
| I₂ (Aromatic Protons) | 7.25-6.60 |
| I₃ (Olefinic Protons-Type 2) | 6.60-5.95 |
| I₄ (Olefinic Protons-Type 1) | 5.95-5.67 |
| I₅ (Olefinic Protons-Type 2) | 5.67-5.35 |
| I₆ (Olefinic Protons-Type 3) | 5.35-5.15 |
| I₇ (Olefinic Protons-Type 1) | 5.15-4.85 |
| I₈ (Olefinic Protons-Type 4) | 4.85-4.40 |
| I₉ (Paraffinic Protons) | 4.40-0.25 |

The listed types of olefinic protons were assumed to correspond to the following structures:

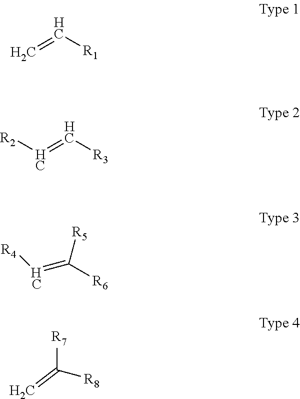

The amounts of aromatic, olefinic and aliphatic protons were determined based on the assigned peak integrals according to the following equations:

$$Mol \% Aromatic Protons = [ ( {I}_{1} + {I}_{2} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 1 = [ ( {I}_{4} + {I}_{7} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 2 = [ ( {I}_{3} + {I}_{5} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 3 = [ ( {I}_{6} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 4 = [ ( {I}_{8} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Paraffinic Protons = [ ( {I}_{9} ) / ( {I}_{1} + {I}_{2} + {I}_{3} + {I}_{4} + {I}_{5} + {I}_{6} + {I}_{7} + {I}_{8} + {I}_{9} ) ] \%$$

6) The water content of the catalyst was determined using a Sartorius MA45 (Sartorius AG, Goettingen, Germany) on a sample of 0.5 to 1 g at 180° C.
7) For the determination of a pH value of the hydrodepolymerization products, a liquid sample of the hydrodepolymerization product was extracted with water in a volume ratio water:sample of 1:5 The pH value of the aqueous solution was measured.
8) Particle size distribution of the particulate non-porous support and the catalyst were determined according to Coulter counter analysis in accordance with ASTM D4438.
9) Properties of the organic waste material were determined as follows:

Samples from 20 to 100 g of the waste material were milled and analyzed. Alternatively, a pelletized sample of the polymeric waste was analyzed.

The following methods are used:
i) Total Volatiles (TV) were measured as the weight loss of a 10 g sample at 100° C. and after 2 hours at 200 mbar.
ii) Water content was determined by Karl-Fischer titration, using an apparatus from Metrohm 915 KF Ti-Touch equipped with a PT100 indicator electrode for volumetric KF titration, according to Metrohm Application Bulletin 77/3e in compliance with ASTM E203.
iii) IR-Spectroscopy was used for a qualitative identification of various polymers (PP, PE, PS, PA, PET, PU, and Polyester) and additives such as CaCO₃
iv) Standard elemental analysis was used for determination of wt. % of H, C, N (DIN 51732: 2014-07) and S (tube furnace, ELTRA GmbH, Haan, Germany, DIN 51724-3: 2012-07).
v) ¹H-NMR was used for determining the composition of polymers soluble in solvents for recording a ¹H-NMR spectrum: PE/PP balance (copolymers were also included), PET, PS.
vi) Ash Content analysis of plastics was determined at 800° C. according to DIN EN ISO 3451-1 (2019-05).
vii) Bulk density of the polymer waste was determined according to DIN 53466.
viii) Corrosivity was determined as the pH value of an aqueous solution after a contact time of 3 h (5 g sample in 50 ml distilled water).
ix) Inductively coupled plasma atomic emission spectroscopy (ICP-AES) was used for quantitative element determination (total chlorine content, content of Si or metals)

Various catalysts were prepared and tested in depolymerizations of different polymeric waste materials.

As the particulate, non-porous support of the catalyst, sand, having a particle size distribution as summarized in Table 2, was employed.

TABLE 2
Particle size distribution

| | Sand wt % |
|---|---|
| >1400 μm | 40.55% |
| >1400 μm->1000 μm | 55.30% |
| <1000 μm | 4.15% |

with 99% of the particles being smaller than 3 mm. The sand employed was pre-dried at 80° C. for 24 h in a drying oven with circulating air.

### Catalyst #4:

The acidic compound was mixed with 25 kg of sand, thereby obtaining a catalyst as shown in Table 4.

### Catalysts #3:

25.0 kg of sand were placed into a 60 L steel barrel with screw cap and equipped with a Teflon inlay. 350 ml mineral oil were added (corresponding to 1.4 wt % with respect to sand). The drum was placed on a Drum Hoop Mixer and rotated for 1 hour (about 100 rpm). 24.5 kg of the resulting mixture was placed in another drum. 500 g of the corresponding acidic component (corresponding to a 2 wt % loading) were added. The drum was placed on a drum hoop mixer and rotated for 1 hour (about 100 rpm). At the end of the mixing process, a free-flowing catalyst was obtained with an even distribution of the particles of the acidic component on the surface of the sand particles.

As the mineral oil, Ondina X 432 was used, which was commercially available from Shell. The mineral oil had the following properties:

TABLE 3

| Property | Method | Shell Ondina X 432 |
|---|---|---|
| Kinematic Viscosity at 20° C. in mm²/s | ISO 3104 | 165 |
| Kinematic Viscosity at 40° C. in mm²/s | ISO 3104 | 59 |
| Kinematic Viscosity at 100° C. in mm²/s | ISO 3104 | 9.0 |

### Preparation of Catalysts #1-#2

The catalyst, using silica hydrogel as coating agent, was prepared as follows:
25.0 kg of sand were placed into a 60 L steel barrel with screw cap and equipped with a Teflon inlay. 500 ml water were added (corresponding to 2.0 wt % with respect to sand). The drum was placed on a Drum Hoop Mixer and rotated for 1 hour (about 100 rpm). 24.5 kg of the resulting mixture was placed in another drum. 1000 g of a 1:1 milled free-flowing mixture of silica hydrogel and the acidic compound (corresponding to a 2 wt % loading) were added. The drum was placed on a drum hoop mixer and rotated for 1 hour (about 100 rpm). At the end of the mixing process, a free-flowing catalyst was obtained with an even distribution of the particles of the acidic compound on the surface of the sand particles. The resulting mixture was dried at 120° C. vacuum for 6 h.

The silica hydrogel was prepared according to EP1290042, example 1. The solid content of the hydrogel sample was 20 wt %. The D50 of the milled mixture of silica hydrogel and acidic components were between 80-100 μm, in accordance with ASTM D4438.

Table 4 summarizes the catalysts employed with the amount of the acidic compound given in wt.-% with respect to sand.

TABLE 4

| Catalyst | | | amount acidic |
|---|---|---|---|
| # | coating agent | acidic compound | compound |
| 1 | silica hydrogel | Zeolyst Beta | 2 |
| 2 | silica hydrogel | Zeolyst ZSM-5 | 2 |
| 3 | oil | Zeolyst Beta | 2 |
| 4 | none | Zeolyst Beta | 2 |

### Acidic Compounds:

Zeolyst ZSM-5 and Zeolyst Beta (CP811E-75) were commercially available from PQ Corporation, Malvern, PA, USA

### Feedstock:

The following organic waste materials were employed as feedstocks:
A) Shredded Multilayer PEX pipe waste with an Aluminum layer (Particle size<20 mm)
B) Shredded PEX pipe waste with EVOH (Particle size<20 mm)
C) Shredded PEX pipe waste with an Aluminum layer (Particle size<20 mm)
D) Shredded monolayer PEX pipe waste (Particle size<20 mm)
E) Shredded high voltage cable crosslinked waste (Particle size<20 mm)
F) Shredded and pelletized mixed plastic waste from household packaging (comparison)
G) 1:1 mixture of feedstock D) and a post-consumer recyclate with an MFR (21.6) of 20 g/10 min blended at 190° C., thereby obtaining extrudable pellets of MFR (21.6) of 15 g/10 min.

The properties of the feedstocks averaged on analysis of three samples are summarized in Table 5.

TABLE 5

| | | | | | | Polymer composition analysis | | | | | |
|---|---|---|---|---|---|---|---|---|---|---|---|
| | | | | | | | | | | | Other |
| | Ash | TV | BD | Cl | PE + PP | PE | PP | PET | PS | PA | cont. |
| Feed-stock | wt % | wt % | g/cm³ | wt % | wt % | wt % | wt % | wt % | wt % | wt % | wt % |
| A | 25% | 0.5 | 356 | <0.01 | 75 | ~75 | — | — | — | — | 25 |
| B | 0.5 | 1 | 316 | <0.01 | | >95 | — | — | — | — | 2 |
| C | 11.2 | 1.5 | 326 | <0.01 | | >87 | — | — | — | — | 13 |
| D | <0.1 | 1.7 | 360 | <0.01 | | >98 | — | — | — | — | — |
| E | 6.5 | 0.6 | 480 | <0.01 | | >93 | — | — | — | — | 7 |
| F | 4.8 | 4 | 230 | 0.2 | 88 | 72 | 16 | 2.3 | 1.2 | — | 8.5 |
| G | <0.1 | 0.3 | 529 | <0.01 | | >98 | — | — | — | — | — |

Ash: ash content
TV: total volatiles
BD: bulk density
Cl: total chlorine content
PE: polyethylene content
PP: polypropylene content
PET: content of polyethylene terephthalate
PS: polystyrene content
PA: polyamide content
Other cont.: content of other contaminants

The feedstock and catalyst were introduced into a reactor with a screw conveyor at 450° C. and 30 min residence time. The product compositions of the depolymerization process are summarized in Table 6. The resulting gaseous fractions were further separated into liquid and gaseous depolymerization products by condensation. The amounts of the resulting fractions are also given in Table 6.

TABLE 6
Process parameter and mass balance

| Run# | Cat# | Feedstock | % Wax¹ | % Liquid²⁾ | % H₂O | % Gas | % Residue | % loss |
|---|---|---|---|---|---|---|---|---|
| 1 | 1 | A | 0 | 11.7 | 0.7 | 60.3 | 28.4 | −1.1 |
| 2 | 1 | B | 0 | 20.3 | 0.9 | 82 | 0 | −3.2 |
| 3 | 1 | C | 0 | 13.2 | 0.6 | 77.4 | 12.1 | −3.3 |
| 4 | 1 | D | 0 | 18.5 | 0.8 | 81.7 | 1.9 | −2.9 |
| 5 | 1 | E | 0 | 16.6 | 0.9 | 74.4 | 7.2 | 0.9 |
| C6 | 1 | F | 0 | 36.2 | — | 52.9 | 8.9 | 1.9 |
| C7 | 4 | F | (0) | 46.7*⁾ | — | 32.4 | 10.1 | 10.7 |
| C8 | no Cat. | F | (0) | 52.4*⁾ | — | 29 | 18 | 1 |

¹solid at room temperature;
²⁾liquid at room temperature
*⁾visible yellowish particles after cooling, liquid fraction turned to a suspension containing waxes

Runs #1-5 demonstrate depolymerization of crosslinked plastic waste, with over 70% of gas generation with hydrocarbon content of more than 95%. The resulting HC liquids show low viscosity and a brown/red color. In contrast, the resulting HC liquids of runs #C7-C8 were black colored with high viscosity and visible wax particles. Low amount of methane, CO and CO₂ were obtained in exemplar runs #1-5, thereby demonstrating high selectivity of the catalyst towards olefins.

Run #4 was repeated with Feedstock G. Results to feedstock D were obtained.

In Run #C7, a high amount of loss due to reactor fouling was observed.

The resulting residue/sand mixture from Runs #1 and #3 was sieved over a 2 mm grid. The pieces of aluminum layer were separated from the sand/residue mixture, thereby enabling further recycling of aluminum content of the PEX pipe of up to 90% of the total Al content even by simple sieving.

In Run #1, 18% Al yield, and in Run #3, 9% Al yield, based on starting Feedstock A respectively, were recovered.

Comparative runs with the polymeric waste material with bulk density of 60 respectively 150 g/cm³ were carried out in the same reactor set-up. The runs were impacted repeatedly by blockage of feeding line and reactor fouling which rendered the runs troublesome.

Table 7 shows the mass balance of the gaseous depolymerization product obtained in Runs #1-8.

TABLE 7
mass balance of the gaseous depolymerization product

| | Run | | | | | | | |
|---|---|---|---|---|---|---|---|---|
| | 1 | 2 | 3 | 4 | 5 | C6 | C7 | C8 |
| | Feedstock | | | | | | | |
| | A | B | C | D | E | F | F | F |
| | Catalyst # | | | | | | | |
| | 1 | 1 | 1 | 1 | 1 | 1 | 4 | no Cat. |
| Components | Wt.-% | Wt.-% | Wt.-% | Wt.-% | Wt.-% | Wt.-% | Wt.-% | Wt.-% |
| H₂ | 0 | 0.1 | 0.1 | 0.1 | 0.1 | 0.0 | 0.0 | 0.0 |
| CO | 0.6 | 0 | 0 | 0.0 | 0.0 | 11.4 | 11.3 | 6.4 |
| CO₂ | 3.7 | 0.6 | 0.5 | 0.5 | 2.0 | 22.6 | 22.3 | 36.5 |
| CH₄ | 0.5 | 0 | 0 | 0.0 | 0.6 | 5.5 | 5.4 | 3.6 |
| C₂H₆ | 0.4 | 0.6 | 0.7 | 0.5 | 0.4 | 7.6 | 7.5 | 5.8 |
| C₂H₄ | 1.1 | 1.4 | 1.5 | 2.0 | 1.6 | 6.8 | 6.7 | 5.1 |
| C₃H₈ | 3.4 | 2.7 | 3.2 | 3.4 | 0.7 | 7.1 | 7.0 | 4.4 |
| C₃H₆ | 34.6 | 28.8 | 28.3 | 29.6 | 32.1 | 15.1 | 14.9 | 15.6 |
| Butanes | 27.9 | 23.4 | 22.6 | 23.2 | 22.5 | 3.6 | 3.5 | 1.8 |
| Butenes | 27.7 | 42.4 | 43.2 | 40.7 | 40.0 | 8.9 | 8.9 | 10.8 |
| Rest | 0 | 0 | 0 | 0.0 | 0.0 | 11.4 | 12.5 | 10.2 |
| Olefins | 63.4 | 72.6 | 73.0 | 72.3 | 73.7 | 30.8 | 30.5 | 31.4 |
| total HC | 95.6 | 99.3 | 99.5 | 99.4 | 97.9 | 54.6 | 54.0 | 46.9 |
| Olefins/HC | 66.3 | 73.1 | 73.4 | 72.7 | 75.3 | 56.3 | 56.4 | 66.9 |

1. A method for producing a cracker feedstock by depolymerizing a polymeric waste material comprising crosslinked polyethylene (PEX), comprising the steps of:
i) introducing the waste material into a pyrolysis reactor;
ii) mixing the waste material with a depolymerization catalyst comprising an acidic compound;
iii) depolymerizing the waste material in an oxygen-free atmosphere at a temperature ranging from 400 to 590° C., thereby obtaining a gaseous depolymerization product; and
iv) collecting and condensing at least part of the gaseous depolymerization product, thereby obtaining the cracker feedstock.

2. The method of claim 1, wherein the acidic compound is deposited on a particulate non-porous support with the aid of a coating agent.

3. The method of claim 2, wherein the acidic compound is selected from the group consisting of Al/Si mixed oxides, Al₂O₃, aluminosilicates, silica and zeolites.

4. The method of claim 2, wherein the particulate non-porous support is selected from the group consisting of sand, glass beads and metal particles and the coating agent is selected from the group consisting of oil, inorganic hydrogel and combinations thereof.

5. The method of claim 1, wherein the waste material further comprises aluminum and the method further comprising a step of: collecting the aluminum from the waste material.

6. The method of claim 1, wherein the polymeric waste material comprises a polymeric fraction comprising an amount of non-polyolefin components lower than 12 wt. %, based on the total weight of the dry weight polymeric fraction of the waste material feedstock.

7. The method of claim 1, wherein the polymeric waste material has a bulk density of at least 150 g/cm³, determined according to DIN 53466.

8. The method of claim 1, wherein the crosslinked polyethylene is selected from the group consisting of peroxide-crosslinked polyethylene, silane-crosslinked polyethylene, irradiation-crosslinked polyethylene, radically crosslinked polyethylene, azo-crosslinked polyethylene, UV initiated radically crosslinked polyethylene and mixtures thereof.

9. The method of claim 1, wherein the polymeric waste material is obtained from PEX pipes, PEX/Al/PEX pipes, PEX/Al/PE-RT pipes, PEX/Al/PE pipes, or combinations thereof.

10. The method of claim 1, wherein the polymeric waste material comprises shredded PEX pipe waste and plastic waste stream with a melt index (MFI/21.6) of higher than 10 g/10 min.

11. The method of claim 1, wherein the pyrolysis reactor is a reactor with a screw conveyer and the gaseous depolymerization product is released from the reactor by gas release units placed along the screw conveyer.

12. The method of claim 1, wherein the residence time of the waste material in the reactor is no more than 60 minutes.

13. The method of claim 1, wherein the reactor is operated at a pressure of 0.7 to 10 bar_{g}.

14. The method of claim 1, wherein the collected depolymerization product comprises more than 30 wt.-% of gaseous components, based on the weight of polymeric fraction of the polymeric waste material.

15. The method of claim 1, wherein the gaseous depolymerization product comprises a depolymerization liquid product comprising from 35 to 45 wt.-% of a high boiling fraction, from 40 to 50 wt.-% of a medium boiling fraction, and from 15 to 25 wt.-% of a low boiling fraction.
